(19) United States
(12) Patent Application Publication
(10) Pub. No.: US 2008/0031913 A1
(43) Pub. Date: Feb. 7, 2008

(54) METHOD FOR MANUFACTURING BIOMEDICAL FILLER MATERIAL RICH-CONTAINED WITH GROWTH FACTOR

(75) Inventor: YU-YU TSAI, TAIPEI (TW)

Correspondence Address: ROSENBERG, KLEIN & LEE, 3458 ELLICOTT CENTER DRIVE-SUITE 101, ELLICOTT CITY, MD, 21043, US

(73) Assignee: NAN-HUEI YEH, TAIPEI (TW)

(21) Appl. No.: 11/833,604

(22) Filed: Aug. 3, 2007

(30) Foreign Application Priority Data
Aug. 4, 2006 (TW) 095128729

(51) Int. Cl. A61K 9/00; A61F 2/02; A61P 43/00; A61K 38/48

(52) U.S. Cl. 424/422; 424/94.64

(57) ABSTRACT

A method for manufacturing at least one kind of biomedical filler materials rich-contained with at least one growth factor is disclosed, wherein at least one dry powder composed of the growth factor is provided to cover the surface of a plurality of filler materials via a physical electro-adsorption action. The filler materials have different intensities of electrostatic charge on their surfaces; therefore, with different values of electrovalence, the dry powder can be adsorbed to the surface of the filler materials via the physical electro-adsorption action, so as to obtain the biomedical filler materials rich-contained with the thrombin and the growth factor.

### FIELD OF THE INVENTION

The present invention relates to a method for manufacturing biomedical filler materials, and more particularly to a method for manufacturing biomedical filler materials, which are rich-contained with at least one thrombin factor and at least one growth factor.

### BACKGROUND OF THE INVENTION

Growth factors includes transforming growth factor p (TGF-p), bone-matrix protein (BMP), demineralized bone matrix (DBM), base fibroblast growth factor, platelet generated growth factor and another adipose growth factor. Platelets are generated from megakaryocytes. When blood vessels are injured, α granules within platelet cytoplasm are activated to release the contains thereof, such as von Willebrand factor and fibrinogen. The contains of α granules within the platelet cytoplasm can promote the platelets to adsorb to the wall of the blood vessels and form the platelet aggregations, so as to stanch the blood.

The α granules can be activated to release the growth factors, such as platelet derived growth factor (PDGF), transforming growth factor β (TGF-β), vascular endothelial growth factor (VEGF), epithelial growth factor (EGF), insulin-like growth factor (IGF) ,etc. When wounds are healed, the growth factors can promote the regeneration, movement and differentiation of cells, and the growth factors can further promote the synthesis of collagen, the mobility of blood vessels. Nevertheless, it has been certified that the growth factors can be mixed with platelet-rich plasma (PRP) extracted from blood to promote the regeneration of the bones after the medical operations.

PRP is a kind of material, which is extracted from a patient's blood and layer-divided after suffering a centrifugal condition, so as to contain a high-concentration of platelet for stanching the blood. PRP further contains many kinds of growth factors, so that PRP are also helpful for bone regeneration in combination with the utilization of auto-graft. The dry thrombin factors are mainly obtained from PDGF of platelet, or extracted from bone matrix. Additionally, other cells, such as macrophages, smooth cells neighbor to blood vessels, and endothelial cells, etc., also can secrete PDGF. PDGF is a kind of polypeptide growth factors, which can activate a series of biochemical reactions with at least one specific receptor. The actions of the biochemical reactions can be observed in three aspects of: the influence of bone regeneration, the action to periodontal ligament fibroblast, and the condition of combining the periodontal regeneration operation. Moreover, PDGF can further promote the regeneration of bone cells and the production of collagen.

Furthermore, in the present, most biomedical filler materials are provided with inorganic calcium salt served as the substrate, so that it can control the dissolution rate via controlling the composition of the biomedical filler materials materials, or execute bone-integration via bone growth factors. However, in the conventional method, it is necessary to do complicated mixing procedure when an operation is in progress, so as to cause more risk.

Therefore, from above description, in practice, it is apparent that the conventional filler materials existing inconvenience and shortcomings need to be improved. Thus, according to related experience and combining with the theoretical application, the inventor engages in numerous observations and researches to improve above inconvenience and shortcomings, so as to disclose the present invention under a reasonable design for improving above shortcomings.

### SUMMARY OF THE INVENTION

The primary objective of the present invention provides a method for manufacturing medical filler materials rich-contained with at least one thrombin factor and at least one growth factor. In the method, at least one dry powder composed of the growth factor is provided to cover the surface of a plurality of filler materials via a physical electronic-adsorption action. The filler materials have different intensities of electrostatic charge on their surfaces; therefore, with different values of electrovalence, the dry powder can be adsorbed to the surface of the filler materials via the physical electronic-adsorption action, so as to obtain the biomedical filler materials rich-contained with the growth factor.

The second objective of the present invention provides a method for manufacturing medical filler materials rich-contained with at least one thrombin factor and at least one growth factor. In the method, the filler materials can be bone-grafts, such as calcium sulfate salt, calcium phosphates salt, etc., which are capable of being absorbed by human's body.

The third objective of the present invention provides a method for manufacturing medical filler materials rich-contained with at least one thrombin factor and at least one growth factor. In the method, the filler materials are characterized in having an appearance with powder composed of particles with different sizes; as time going, to form biomedical filler materials, which have specified strengths after mixing with different mixed-liquids.

The fourth objective of the present invention provided a method for manufacturing medical filler materials rich-contained with at least one thrombin factor and at least one growth factor. In the method, growth factors for bone directly cover the surface of bone-grafts via a physical electro-adsorption action. Thus, it is not necessary to do complicated mixing procedure when an operation is in progress, so as to make the operation can be executed more quickly and decrease the risk of the failure of the operation.

For achieving above objectives, the present invention provides a method for manufacturing biomedical filler materials rich-contained with at least one thrombin factor and at least one growth factor. The method comprises the steps of: providing a reaction container; providing a plurality of filler materials, and executing a classification, blending toward the filler materials; covering the filler materials with a dry first powder composed of the thrombin factor, and depositing the filler materials into the reaction container; and mixing a mixed-liquid with a second powder rich-contained with the growth factor to obtain a mixture, and further homogeneously mixing the mixture with the filler materials covered with the dry first powder, so as to obtain the biomedical filler materials rich-contained with the thrombin factor and the growth factor.

The means, characteristics, and the preferred embodiment of this invention are described with relative figures as follows.

### BRIEF DESCRIPTION OF THE DRAWINGS

The technical means adopted by the present invention to achieve the above and other objects can be best understood by referring to the following detailed description of the preferred embodiments and the accompanying drawings, wherein

FIG. 1 is a flowchart illustrating the method for manufacturing biomedical filler materials rich-contained with thrombin factor and growth factor;

FIG. 2 is an X-ray diffraction diagram of calcium sulfate hemihydrate; and

FIG. 3 is an X-ray diffraction diagram of a second powder rich-contained with at least one growth factor.

### DETAILED DESCRIPTION OF THE PREFERRED EMBODIMENTS

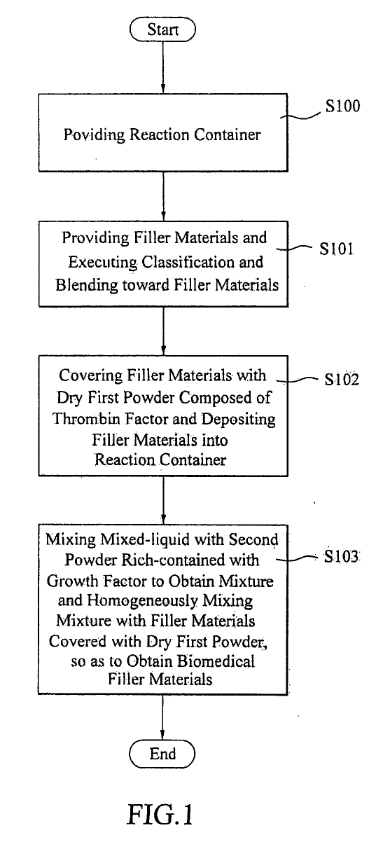

With reference to FIG. 1, the present invention provides a method for manufacturing biomedical filler materials rich-contained with at least one thrombin factor and at least one growth factor. The method comprises the steps of: providing a reaction container (Step 100); providing a plurality of filler materials, and executing a classification and blending toward the filler materials (Step 101); covering the filler materials with a dry first powder composed of the thrombin factor, and depositing the filler materials into the reaction container (Step 102); and mixing a mixed-liquid with a second powder rich-contained with the growth factor to obtain a mixture, and further homogeneously mixing the mixture with the filler materials covered with the dry first powder, so as to obtain the biomedical filler materials rich-contained with the thrombin factor and the growth factor (Step 103). The reaction container is made of acrylic material. The filler materials can be bone-grafts, such as calcium sulfate salts, calcium phosphates salts, etc., which are capable of being absorbed by human's body. The filler materials can be provided in one of a tablet condition, a powder condition and a colloid condition. When the filler materials are provided in the powder condition, the filler materials are composed of powder particles size mainly within a distribution range of 50 to 320 meshes.

In Step 102, the dry first powder composed of the thrombin factor covers the surface of the filler materials via a physical electronic-adsorption action. The mixed-liquid is selected in one of platelet-rich plasma (PRP) made from a blood after suffering an ultra-centrifugal condition, normal saline, medical-injection water and medical-grade dilute phosphoric acid. The second powder rich-contained with the growth factor contains 10% wt (percentage in weight) to 90% wt of thrombin. A mixing proportion of the mixed liquid and the second powder is between 1:0.1 and 1:0.5, wherein the most optimized value of the mixing proportion is 2:1, i.e., the mixing proportion of 1:0.5 is the most optimized.

The first preferred embodiment of the present invention comprises the steps as follows:
(1) executing classification, blending toward calcium sulfate hemihydrate powder provided by J. T. Baker, following ASTM standard of U.S.A., so as to classification, blending the calcium sulfate hemihydrate powder composed of powder particles size range mainly of 200 meshes;
(2) operating a vacuum valve to provide a working pressure of 10⁻³ to 10⁻² torr, and providing a working voltage of 5 kev, so as to make the dry first powder composed of the thrombin factor adsorb to calcium sulfate hemihydrate powder via the physical electro-adsorption action;
(3) providing the reaction container (made of acrylic material) having at least one recessed space with a length of 5 cm, a width of 2 cm and a depth of 2 cm;
(4) providing medical-grade injection water and a stirrer, mixing the medical-grade injection water respectively with second powder (rich-contained with the growth factor) and pure calcium sulfate hemihydrate powder in the mixing proportion of 2:1 to get a first mixture and a second mixture, putting the first mixture and the second mixture into the recessed space of the reaction container, and stirring the stirrer in a rate of 30 revolution per minute (RPM) for 15 seconds to make the first mixture and second mixture within the recessed space be sufficiently mixed; and

(5) preparing the Vicat needle, with a tip area of 1 mm² and a weight of 300 g, served as test equipment, laying the tip of the Vicat needle on the first mixture and second mixture in each interval of 30 seconds until no recessed mark appears on the surface of the first mixture and second mixture, and respectively recording the how many intervals spent until no recessed mark appears on the surface of the first mixture and second mixture, so as to respectively get a first hardening time and a second hardening time.

TABLE 1

| | | Weight of Second | Volume of | Status |
|---|---|---|---|---|
| First | Mixing | Powder and Pure | Medical-grade | after |
| Preferred | Pro- | Calcium Sulfate | Injection | Stirring 15 |
| Embodiment | portion | Hemihydrate | Water | Seconds |
| Second | 2:1 | 10 g | 6 cc | Paste |
| Powder | | | | |
| pure Calcium | 2:1 | 10 g | 6 cc | Paste |
| Sulfate | | | | |
| Hemihydrate | | | | |

TABLE 2

| | Time (in minute) | | | | | | | |
|---|---|---|---|---|---|---|---|---|
| | 1 | 3 | 5 | 7 | 9 | 11 | 13 | 15 |
| Second | X | X | ◯ | ◯ | ◯ | ⊚ | □ | □ |
| Powder | | | | | | | | |
| Pure Calcium | X | X | X | X | ◯ | ◯ | ⊚ | ⊚ |
| Sulfate | | | | | | | | |
| Hemihydrate | | | | | | | | |

Remarks:
X: Thoroughly Perforated;
◯: Partially Perforated;
⊚: Having Some Resistance, but Still Partially Perforated
□: Thoroughly Hardened/Not Perforated

Please refer to Table 1, in initial, both the first mixture and the second mixture are in paste status after stirring for 15 seconds; however, the hardening degree of the first mixture is much different from that of the second mixture. It is obviously that second powder rich-contained with the growth factor has greater hardness.

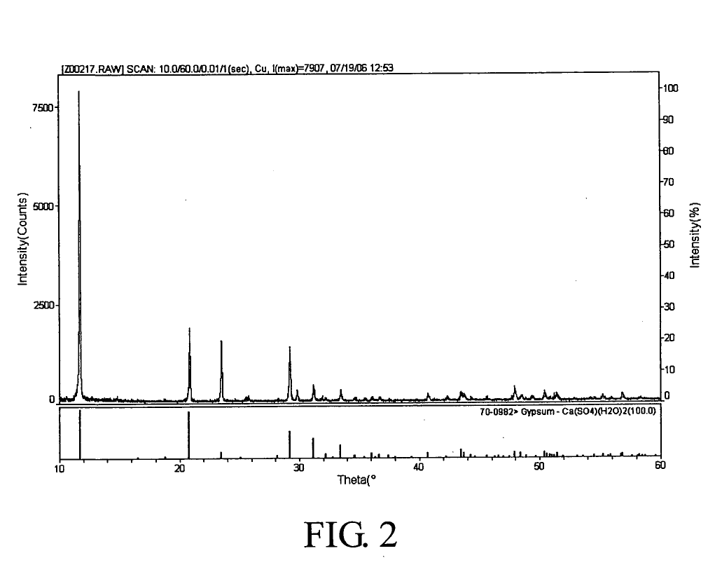

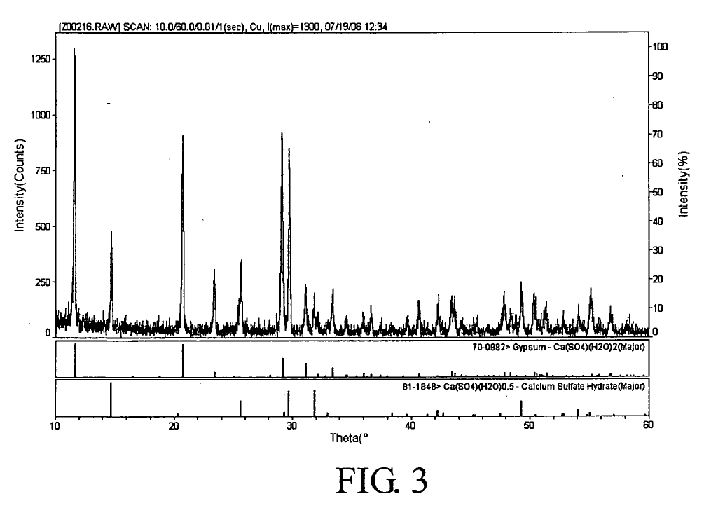

Please further refer to FIG. 2 and FIG. 3, which illustrate two X-Ray experiments respectively toward pure calcium sulfate hemihydrate and the second powder rich-contained with the growth factor. It is obvious that there is only one simplified peak for pure calcium sulfate hemihydrate; while, there are many different peaks for the second powder rich-contained with the growth factor as shown in FIG. 3.

The second preferred embodiment of the present invention comprises the steps as follows:
(1) executing classification, blending toward calcium phosphate powder provided by J. T. Baker, following ASTM standard of U.S.A., so as to classification blending the calcium phosphate powder composed of powder particles size mainly of 200 meshes;
(2) operating a vacuum valve to provide a working pressure of 10-3 to 10-2 torr therein, and providing a working voltage of 5 kev, so as to make the dry first powder composed of the thrombin factor adsorb to calcium sulfate hemihydrate via the physical electronic-adsorption action;
(3) providing the reaction container (made of acrylic material) having at least one recessed space with a length of 5 cm, a width of 2 cm and a depth of 2 cm;
(4) providing medical-grade injection water and a stirrer, mixing the medical-grade injection water respectively with second powder (rich-contained with the growth factor) and pure calcium sulfate hemihydrate in the mixing proportion of 2:1 to get a first mixture and a second mixture, putting the first mixture and the second mixture into the recessed space of the reaction container, and stirring the stirrer in a rate of 30 revolution per minute (RPM) for 15 seconds to make the first mixture and second mixture within the recessed space be sufficiently mixed; and
(5) Preparing the Vicat needle, with a tip area of 1 mm² and a weight of 300 g, served as test equipment, laying the tip of the Vicat needle on the first mixture and second mixture in each interval of 30 seconds until no recessed mark appears on the surface of the first mixture and second mixture, and respectively recording the how many intervals spent until no recessed mark appears on the surface of the first mixture and second mixture, so as to respectively get a first hardening time and a second hardening time.

Summarizing above description, the method for manufacturing the biomedical filler materials, which are rich-contained with the thrombin factor and the growth factor as provided in the present invention, has the advantages of:
(1) adsorbing the dry first powder contained with the thrombin to the surface of the filler materials via the physical electro-adsorption action;
(2) the filler materials are characterized in having an appearance with power composed of particles with different sizes; as time going, to form biomedical filler materials, which have specified strengths after mixing with different mixed-liquids; and
(3) it is not necessary to do complicated mixing procedure when an operation is in progress, so as to make the operation can be executed more quickly and decrease the risk of the failure of the operation.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for manufacturing at least one kind of biomedical filler materials rich-contained with at least one thrombin factor and at least one growth factor, and at least comprising four steps of:
(a) providing a reaction container;
(b) providing a plurality of filler materials and executing a classification, blending toward the filler materials;
(c) covering the filler materials with a dry first powder composed of the thrombin factor, and depositing the filler materials into the reaction container; and
(d) mixing a mixed-liquid with a second powder rich-contained with the growth factor to obtain a mixture, and further homogeneously mixing the mixture with the filler materials covered with the dry first powder, so as to obtain the biomedical filler materials rich-contained with at least one thrombin factor and growth factor.

2. The method as claimed in claim 1, wherein the reaction container is made of an acrylic material.

3. The method as claimed in claim 1, wherein the filler materials are made of at least one kind of bone-grafts, which are capable of being absorbed by human's body.

4. The method as claimed in claim 3, wherein each of the bone-grafts is selected from one of a calcium sulfate salt, a calcium phosphate salt, and another inorganic calcium compound capable of being applied in biomedical use.

5. The method as claimed in claim 1, wherein the filler materials are provided in one of a tablet condition, a powder condition and a colloid condition.

6. The method as claimed in claim 5, wherein when the filler materials are provided in the powder condition, the filler materials are composed of powder particles size ranged mainly within a distribution range of 50 to 320 meshes.

7. The method as claimed in claim 1, wherein in the step (c), each of the filler materials has at least one surface covered with the dry first powder composed of the growth factor via a physical electro-adsorption action.

8. The method as claimed in claim 1, wherein the mixed-liquid is selected in one of platelet-rich plasma (PRP) made from blood after suffering an ultra-centrifugal condition, a normal saline, medical-injection water and medical-grade dilute phosphoric acid.

9. The method as claimed in claim 1, wherein the second powder contains 1 0% wt (percentage in weight) to 90% wt of thrombin.

10. The method as claimed in claim 1, wherein a mixing proportion of the mixed liquid and the second powder is between 1:0.1 and 1:0.5.
